(19) United States
(12) Patent Application Publication
(10) Pub. No.: US 2004/0265233 A1
(43) Pub. Date: Dec. 30, 2004

(54) Composite particle containing superparamagnetic iron oxide

(76) Inventors: Susanne Holzer, Ottweiler (DE); Wolfgang Krause, Kirkel-Limbach (DE); Christoph Lesniak, Buchenberg (DE); Helmut Schmidt, Saarbrucken-Gudingen (DE)

Correspondence Address: GREENBLUM & BERNSTEIN, P.L.C., 1950 ROLAND CLARKE PLACE, RESTON, VA, 20191, US

(21) Appl. No.: 10/493,063

(22) PCT Filed: Oct. 30, 2002
(86) PCT No.: PCT/EP02/12118

(30) Foreign Application Priority Data
Oct. 31, 2001 (DE) 101 53 639.9

(51) Int. Cl. A61K 49/00; B29C 39/10

(52) U.S. Cl. 424/9.32; 264/4

(57) ABSTRACT

A method for producing composite particles in which superparamagnetic iron oxide particles having a diameter of less than 30 nm are contained in a polysiloxane matrix comprising functional groups. The composite particles obtained by the method are suitable for magnetic separation methods.

The present invention relates to composite particles which comprise superparamagnetic iron oxide particles which have a particle diameter of less than 30 nm and are incorporated in a polysiloxane matrix having functional groups, and to a process for their preparation. The composite particles are suitable for magnetic separation.

The use of composite particles in separation systems is known. For this purpose, composite particles are used in which ferromagnetic particles are incorporated into an organic polymer matrix having amino, carboxyl or chelate functions or into a silicate matrix, or the composite particles have a nonmagnetic core such as glass or plastic which is coated with various shells such as FeOₓ.

The present invention provides a process for preparing composite particles which comprise superparamagnetic iron oxide particles having a particle diameter of less than 30 nm which are incorporated in a polysiloxane matrix, in particular a polyorganosiloxane matrix, having functional groups, by condensing a precondensate obtained from one or more hydrolyzable silane compounds in an aqueous-organic emulsion which comprises the iron oxide particles and the precondensate to form the polysiloxane matrix and optionally removing the resulting composite particles, at least one hydrolyzable silane compound used having at least one functional group and/or a reaction with at least one organic compound which has at least one functional group, in particular a hydrolyzable silane compound, being effected in a later reaction step. It is possible by the process to obtain the inventive composite particles which comprise superparamagnetic iron oxide particles having a particle diameter of less than 30 nm which are incorporated in a polysiloxane matrix having functional groups.

The superparamagnetic composite particles obtainable by the process according to the invention offer the advantage that various functionalizations may be introduced by one and the same synthetic principle as early as in the particle synthesis. This results in a highly flexible superparamagnetic particulate separating system composed of organic-inorganic nanocomposite materials for which the functionalization may be selected flexibly for specific fields of application. The organically modified silane matrix of the inventive superparamagnetic composite particles can be assembled according to the modular principle by the selection of the functionalized compounds, preferably functionalized alkoxysilanes, functioning as the precursor components.

The inventive functionalized superparamagnetic composite particles consist of a functionalized silane matrix into which superparamagnetic iron oxide single-domain particles are embedded. The iron oxide particles are mixed with the matrix precursors (hydrolyzable silanes, especially alkoxysilanes) in a W/O emulsion and the matrix components are preferably condensed by evaporating the aqueous phase, in particular by adding the emulsion dropwise to a hot solvent (emulsion evaporation).

In the process, superparamagnetic composite particles having average diameters of preferably 100 nm-2 μm are prepared. The content of superparamagnetic iron oxide particles in the composite can be used to vary the specific magnetization. The use of different functionalized alkoxysilanes provides composite particles to which functionalities are bonded covalently which are suitable for adsorption/complexation of different substance groups, or to which further compounds having specific affinity for certain substances/substance groups may be coupled. To the inventive composite particles may be coupled biomolecules such as proteins, enzymes (catalytic properties) or antibodies, so that they may also be used in the biochemical field.

In the process, preference is given to using superparamagnetic amino-functionalized FeOₓ single-domain particles which are flocculation-stable in the acidic pH range and are preferably introduced into the aqueous phase of a W/O emulsion, and an acid-pretreated sol of the matrix precursors (for example tetra(m)ethoxysilane and a functionalized trialkoxysilane) is subsequently added. The matrix precursors are condensed to give the solid matrix by emulsion evaporation and the FeOₓ single-domain particles are fixed in the functionalized matrix. Particular preference is given to using amino-functionalized superparamagnetic iron oxide particles whose preparation is described in EP-B-892834 which is incorporated herein by reference. It is possible to selectively prepare superparamagnetic composite particles whose saturation magnetization is varied via the content of iron oxide nanoparticles. Since the particles exhibit superparamagnetic behavior and thus do not irreversibly aggregate as a consequence of magnetic interactions, they may be used repeatedly as suspended individual particles in an aqueous medium.

The particulate magnetic separation systems have a broad field of application, for example for the removal of the heavy metal ions from aqueous phases or for the recovery of noble metals. To this end, magnetic separation systems have to be equipped flexibly with different specific functionalities, for example with different complex ligands which selectively capture certain ions. The agglomeration of the individual composite particles as a consequence of magnetic dipole interactions which results from permanent magnetization leads to a reduction in the active surface area and to more rapid sedimentation under gravity.

The inventive composite particles function as support components for active components which may be moved, directed and separated in liquid media using magnetic fields. To this end, the superparamagnetic composite particles are coupled with an application-specific functionalization or active component and may be used in a liquid medium as nonagglomerated individual particles, for example for adsorbing harmful substances, cells, or for catalysis or in the support-bound synthesis of organic compounds, and be separated in a magnetic field after the application. The composite particles should have good response to magnetic fields (high specific magnetization), in order to achieve rapid separation.

Superparamagnetic particles are derived from ferro- and ferrimagnetic particles, although the size of superparamagnetic particles is below the size of the magnetic domains (Weiss domains, <30 nm). These are therefore also referred to as single-domain particles. When the intention is to remove single-domain particles from suspensions, high magnetic field strengths (>5 000 oersteds) are required, since these small particles are subject to intense thermal motion.

Since the separation of superparamagnetic single-domain particles requires very strong magnetic fields, they are just as ill-suited for use in magnetic separation processes as larger multidomain particles which can be removed by weak magnetic fields but retain remanent magnetization which leads to the agglomeration of the individual particles, which is a hindrance to reuse of the particles. In the case of the inventive composite particles, a multitude of superparamagnetic iron oxide single-domain particles having a diameter below 30 nm is therefore fixed in a functionalized silane matrix. Composite particles are thus obtained which have a good response to magnetic fields and nevertheless have superparamagnetic properties. The inventive functionalized superparamagnetic composite particles consisting of a functionalized SiO₂ matrix into which the iron oxide nanoparticles (preferably magnetite, maghemite) are embedded may be prepared with average sizes in a nanometer and micrometer range, preferably from 100 nm to 2 μm.

Useful superparamagnetic components in the synthesis are iron oxide particles having average particle diameters below 30 nm, preferably having average diameters of 5-20 nm. The iron oxide nanoparticles used may either be unmodified or have been surface-modified, preferably with alkoxysilanes, especially γ-aminopropyltriethoxysilane. The response of the particles to magnetic fields may be varied via the contents of superparamagnetic iron oxide single-domain particles in the composite particle. At an FeOₓ content of approx. 15% by weight, a specific magnetization of 11.2 EMU/g was achieved. The density of these composite particles is 1.7 g/cm³, so that even composite particles having sizes in the micrometer range sediment only slowly under gravity. Composite particles having a specific magnetization of 21.4 EMU/g were prepared by increasing the FeOₓ content. In the case of a composite having a d₅₀ value of the diameter of 240 nm (80% of the composite particles in the size range of 170 nm-380 nm) a BET surface area of 11.9 m²/g was obtained. The particles may be isolated and stored as a dry powder. They are redispersible and reusable.

Useful superparamagnetic nanoparticles are ferrites and in particular magnetite or maghemite particles which bear no surface modification or have been surface-modified, especially with functionalized alkoxysilanes, preferably γ-aminopropyltriethoxysilane (APS) or N-(2-aminoethyl)-3-aminopropyltrimethoxysilane.

The matrix of the superparamagnetic composite particles is formed by a sol-gel process, preferably from a structure-forming tetraalkoxysilane, preferably tetraethoxysilane TEOS, as a matrix precursor, for example by acidic hydrolysis and subsequent condensation. The surface of composite particles whose matrix has been formed by a structure-forming tetraalkoxysilane alone may optionally also be provided with the desired functionalization in a separate synthetic step, for example via known sol-gel processes.

Examples of functional groups which are present on the hydrolyzable silanes or the organic compound which bears a functional group are amino, alkyl-substituted amino, carboxyl or carboxylate, epoxy, mercapto or mercaptide, cyano, hydroxyl or ammonium groups. A plurality of functional groups may also be present and may then function as chelate formers, for example the derivatives corresponding to ethylenediaminetetraacetic acid. Further examples are listed below for the silanes. The functional groups in the silanes are typically bonded to Si via a hydrocarbon group and constitute the nonhydrolyzable radical having a functional group, as detailed below, although the hydroxyl group may also, for example, be bonded directly to Si.

Generally, hydrolyzable silanes of the general formula (I) may be used:

RₐSiX₍₄₋ₐ₎ (I)

where the R radicals may be the same or different and constitute nonhydrolyzable groups, the X radicals may be the same or different and be hydrolyzable groups or hydroxyl groups, and a may have the values 0, 1, 2 or 3, preferably 0 or 1.

In the general formula (I), the hydrolyzable X groups are, for example, hydrogen, halogen, alkoxy (preferably C₁₋₆-alkoxy, e.g. methoxy, ethoxy, n-propoxy, isopropoxy and butoxy), aryloxy (e.g. phenoxy), acyloxy (preferably C₁₋₆-acyloxy, e.g. acetoxy or propionyloxy), alkylcarbonyl (preferably C₂₋₇-alkylcarbonyl, e.g. acetyl), amino, monoalkylamino or dialkylamino, preferably having from 1 to 12, in particular from 1 to 6, carbon atoms. Preference is given to alkoxy, in particular methoxy and ethoxy.

The nonhydrolyzable R radicals which may be the same or different may be nonhydrolyzable R radicals having a functional group or without a functional group.

The nonhydrolyzable R radical is, for example, alkyl (preferably C₁₋₈-alkyl, such as methyl, ethyl, n-propyl, isopropyl, n-butyl, s-butyl and t-butyl, pentyl, hexyl, octyl or cyclohexyl), alkenyl (preferably C₂₋₆-alkenyl, e.g. vinyl, 1-propenyl, 2-propenyl and butenyl), alkynyl (preferably C₂₋₆-alkynyl, e.g. acetylenyl and propargyl) and aryl (preferably C₆₋₁₀-aryl, e.g. phenyl and naphthyl). The R and X radicals may optionally have one or more typical substituents, e.g. halogen or alkoxy.

Specific examples of the functional groups of the R radical are the epoxy, hydroxyl, ether, amino, monoalkylamino, dialkylamino, amide, carboxyl, vinyl, acryloyl, methacryloyl, cyano, halogen, aldehyde, alkylcarbonyl and phosphoric acid groups. More than one functional group may be present. The functional groups are bonded to the silicon atom via alkylene, alkenylene or arylene bridging groups which may be interrupted by oxygen or NH groups. The bridging groups mentioned are derived, for example, from the abovementioned alkyl, alkenyl or aryl radicals.

To assemble the matrix, preference is given to using a tetraalkoxysilane, preferably tetraethoxysilane (TEOS), or a tetraalkoxysilane is used as a structure former, preferably tetraethoxysilane, and further alkoxysilanes, in particular functionalized trialkoxysilanes, are condensed on in a separate synthetic step via sol-gel processes.

The matrix is preferably formed by cocondensing a tetraalkoxysilane, preferably tetraethoxysilane, with one or more hydrolyzable silanes having at least one functional group, especially functional trialkoxysilanes (RSiX₃ where X=alkoxy and R=nonhydrolyzable radical having a functional group), preferably γ-aminopropyltriethoxysilane, (2-aminoethyl)-3-aminopropyltrimethoxysilane, anions of N-(trimethoxysilylpropyl)ethylenediaminetriacetic acid and 2-cyanoethyltrimethoxysilane.

The superparamagnetic composite particles may be prepared directly with certain functionalities by the use of functionalized alkoxysilanes which have been selected specifically for the application as matrix precursors. The different functionalities are introduced by cocondensing the functionalized alkoxysilane with a structure-forming alkoxysilane, in particular tetraalkoxysilane.

Suitable functionalized matrix precursors are, for example, γ-aminopropyltriethoxysilane (APS) and N-(2-aminoethyl)-3-aminopropyltrimethoxysilane (AEAPS) (amino functionalization), the sodium salt of N-(trimethoxysilylpropyl)ethylenediaminetriacetic acid (complex ligand for metal ions), 2-cyanoethyltrimethoxysilane (nitrile functionalization) or N-(trimethoxysilylpropyl)-N,N,N-trimethylammonium chloride (trimethylammonium functionalization).

In a preferred synthetic principle, alcoholic sols of the hydrolyzable silanes are pretreated by addition of acid, preferably by formic acid, in an acidic medium at temperatures above 30° C., preferably at 60° C. Optionally, water may be added in the course of pretreatment, preferably ≦50 mol % of the alkoxy groups present in the system. The size of the composite particles may be varied, for example, via the duration of the pretreatment of the matrix precursors with formic acid at 60° C. To embed the superparamagnetic iron oxide particles into the silane matrix, the superparamagnetic iron oxide single-domain particles are mixed with hydrolyzable silanes (alkoxysilanes) which have been pretreated, for example, with formic acid in the aqueous phase of a W/O emulsion. In the emulsion, the precursors react further under acidic hydrolysis. The precursors may be pretreated separately or as a mixture. In addition to the pretreated matrix precursors, unpretreated precursors may also be added to the emulsion.

The aqueous-organic emulsion is a customary emulsion known to those skilled in the art, as described, for example, in Ullmanns Encyklopadie der technischen Chemie, for instance in the 4th edition in volume 10 under the heading Emulsions. These may be oil-in-water (O/W) or preferably water-in-oil (W/O) emulsions. It is preferably a microemulsion. Usually, at least four components are present: water, an oily substance, an emulsifier or an emulsifier mixture and a solubilizer. Specific examples may be taken from the above-mentioned reference, which is incorporated herein by reference.

The water droplets of the W/O emulsion predetermine the shape of the later composite particles. The condensation of the matrix is preferably achieved by evaporating the aqueous phase. This is achieved, for example, by adding the emulsion dropwise to a hot solvent at temperatures above 100° C., preferably at 160-170° C., in the course of which the aqueous phase of the emulsion evaporates abruptly and is distilled off. The functionalization of the hydrolyzable silanes is retained at these temperatures. As a consequence of the evaporation, the hydrolyzable silanes condense and form a solid functionalized matrix in which the superparamagnetic iron oxides are fixed. The aqueous phase may in principle also be evaporated by other processes such as spray drying, rotary evaporation or evaporation in a vertical pipe furnace.

In a preferred process, the superparamagnetic nanoparticles and the hydrolyzable silanes functioning as the matrix precursor are mixed in the aqueous phase of a W/O emulsion or before the addition to the emulsion, and the superparamagnetic nanoparticles are preferably fixed in the matrix by evaporating the aqueous phase of the emulsion, preferably by adding the emulsion dropwise to a hot solvent at temperatures of above 100° C.

In a preferred process, the precursors added to the emulsion are not only matrix precursors which have been pretreated by hydrolysis and/or precondensation, but additionally unpretreated hydrolyzable silanes, and the superparamagnetic nanoparticles are fixed in the matrix by cocondensing the precursors by evaporating the aqueous phase of the emulsion, preferably by adding the emulsion dropwise to a hot solvent at temperatures of >100° C.

The superparamagnetic composite particles obtained in accordance with the invention have the feature that the specific magnetization can be varied via the content of iron oxide single-domain particles in the overall particle. The surface-specific properties of the particles can be controlled variably by the use of hydrolyzable silanes having different functionalities, and the average size of the composite particles can be varied in the case of narrow particle size distribution via the duration of the pretreatment of the matrix precursors or via the loading of the aqueous phase of the emulsion with FeOₓ and matrix precursors, and also via various emulsion parameters.

According to the invention, functionalized composite particles, preferably having amino functionalization, can also be prepared in such a way that the alcoholic phase is distilled off from iron oxide nanoparticles and hydrolyzable silanes after alkaline prehydrolysis of the silanes in the alcoholic phase and mixing of the sol with an aqueous suspension of the nanoparticles, and the aqueous sol obtained in this way is stirred into a W/O emulsion which is then subjected, for example, to emulsion evaporation.

In a further preferred embodiment, the composite particles are therefore prepared in such a way that an alcoholic sol of the alkoxysilanes, preferably an equimolar mixture of tetraethoxysilane and N-(2-aminoethyl)-3-aminopropyltrimethoxysilane is prehydrolyzed under alkaline conditions and, after addition of an aqueous suspension of the iron oxide particles and removal of the alcohol, is stirred into a W/O emulsion, and the composite particles are obtained by emulsion evaporation.

It is possible via the emulsion process to selectively prepare superparamagnetic composite particles having certain average particle diameters in the range of 0.1 μm-2 μm and narrow particle size distribution (e.g. 1.6 μm±0.4 μm). The functionalized composite particles do not agglomerate and have a low density (e.g. 1.7 g/cm³ with 15% by weight of FeOₓ) and even particles having diameters in the micrometer range sediment only slowly in aqueous suspensions and can be used without mechanical stirring.

The composite particles may be controlled very flexibly with regard to their surface-specific properties (functionalization, zeta potential), their size and their specific magnetization by varying the precursors and the reaction conditions. Amino groups or carboxyl groups on the particle surface offer the possibility of coupling on further natural or synthetic monomers or polymers having application-specific functionalizations or properties.

Magnetic separation processes are used in the medicinal field, in the biochemical field and in the environmental field. Heavy metal ions are commonly separated using ion exchangers which are incorporated into magnetic filler components.

To the composite particles may be covalently bonded in further reaction steps, for example, biomolecules (enzymes or antibodies). Biochemical applications are cell separation or the separation of DNA, and also the possibility of allowing enzymes (catalytic properties) to act in a medium and, after use, recovering them by magnetic separation or of controlling the enzymatic reaction by metered addition and magnetic separation.

The composite particles may also be used in the field of combinatorial chemistry in syntheses on solid supports, in which case the support-bonded products may be separated using magnetic fields. In the field of synthetic organic chemistry, the inventive functionalized superparamagnetic composite particles may be used by combinatorial principles in support-bonded synthesis (for example peptides, proteins, heterocycles). In this case, the filtration in the purification after each synthetic step is replaced by a magnetic separation, which allows the problem of blocked filters to be avoided.

The inventive composite particles may be used as magnetic carriers in the separation of cations, for example of noble metals or heavy metals, anions or harmful substances. In this case, the desired reusability requires a functionalization which can bind the substances or substance groups to be isolated reversibly to the magnetic composite particle. The superparamagnetic composite particles having chelate complex ligands, preferably N-(trimethoxysilylpropyl)ethylenediaminetriacetic acid, bonded on covalently are suitable for separating heavy metal cations from contaminated water. The control of complex formation and remobilization of the complex heavy metal ions is possible by varying the pH.

A further application possibility is the use as a carrier for substances having catalytic properties. One possibility is enzymes. Superparamagnetic composite particles coated with enzymes could be used for the enzymatic degradation of harmful substances, and the enzymes may be recovered via magnetic separation after their use.

### EXAMPLES

### Example 1

### General Synthesis

For the prehydrolysis of the alkoxysilanes, in a 500 ml Schott bottle, 34.52 g (165.7 mmol) of tetraethoxysilane and optionally 165.7 mmol of a further functionalized alkoxysilane, e.g. 3-aminopropyltriethoxysilane, and also 15.24 g of formic acid, are added to 25.27 g of ethanol and the sealed bottle is stored at 60° C. for between 5 h and 10 d to pretreat the alkoxysilane. The iron oxide particles are mixed with the matrix silanes in the aqueous phase of a W/O emulsion. To this end, 9.52 g of Emulsogen® OG (a polyglycerol oleate, HLB value 3) and 10.80 g of Tween® 80 (polyoxyethylene 20) sorbitan monooleate, HLB value 15) are stirred into 78 g of special-boiling-point petroleum (b.p. 180-220° C.) and subsequently subjected to an ultrasound treatment (disintegrator) for 10 min. Under further ultrasound treatment, 20.47 g of an aqueous suspension of iron oxide nanoparticles (2.37% by weight of Fe₃O₄) are added, in the course of which an emulsion forms (W/O=0.2). The Fe₃O₄ nanoparticles used are particles which have been stabilized with a layer of condensed γ-aminopropyltriethoxysilane. After 10 min, 8 g of matrix sol are added. After a further 10 min, the ultrasound treatment is terminated and the emulsion stirred at room temperature for 24 h. To evaporate the aqueous phase of the emulsion and condense the matrix components, 800 ml of SBPP are heated to 170° C. and the emulsion is added dropwise via a pump, in the course of which the aqueous phase evaporates and is distilled off, and the iron oxide particles are fixed in the condensing matrix. After magnetic separation, the composite particles are repeatedly washed with isopropanol and subsequently with water. Finally, the composite particles are concentrated to the dry powder on a rotary evaporator under reduced pressure at 60° C. The composite particles have an FeOₓ content of 15% by weight. A specific magnetization of 11.2 EMU/g is attained. The density of the particles at this iron oxide content is 1.7 g/cm³.

### Example 2

In a similar manner to example 1, composite particles (contents of FeOₓ nanoparticles of 15% by weight) having silanol functionalization ≡SiOH were prepared. The matrix component is tetraethoxysilane. Water corresponding to 50 mol % of the alkoxy groups present in the system was added to the sol in the course of the prehydrolysis and the sol was stored at 60° C. for 5 h. The average diameter of the composite particles is 193 nm (80% of the composite particles are within the size range of 125 nm-340 nm) and the isoelectric point is at pH 2.64.

### Example 3

In a similar manner to example 1, composite particles (contents of FeOₓ nanoparticles of 15% by weight) having complex ligand functionalization were prepared. The structure-forming matrix component is tetraethoxysilane. The TEOS sol was pretreated at 60° C. in accordance with example 2 and added to the emulsion. After a stirring time of 16 h, 0.75 g of the sodium salt of N-(trimethoxysilylpropyl)ethylenediaminetriacetic acid) dissolved in 0.75 g of H₂O) was additionally added to the emulsion and it was stirred for a further 3 h (molar TEOS: chelate complexing agent ratio=5.0:1.0). The average size of the composite particles is 130 nm (80% of the composite particles are within the 95 nm-250 nm size range). The isoelectric point of the composite particles is at pH 1.6.

### Example 4

In a similar manner to example 3, composite particles were prepared under varying reaction conditions. The TEOS sol was pretreated at 60° C. for 16 h. The N-(trimethoxysilylpropyl)ethylenediaminetriacetic acid chelate complexing agent (0.75 g in 0.75 g of H₂O) was added to the emulsion immediately after the TEOS sol (3.5 g) (molar TEOS: chelate complexing agent ratio=4.4:1.0), and the emulsion was stirred for 23 h and then subjected to evaporation.

### Example 5

### Heavy Metal Separation

The composite particles from example 3 and 4 are suitable in order to remove heavy metal cations from the aqueous phase by magnetic separation. The complexation and the separation of Co²⁺ ions (at pH 8.0) and also their remobilization (at pH 2.3) were carried out. The complexation was monitored with the aid of the color change of murexide. The complexation capacity of the superparamagnetic composite particles was determined after the separation by determining the amount of remobilized Co²⁺ and was found to be 0.2 mmol of heavy metal ions per gram of composite particles in the case of the composite particles from example 3, and was found to be 0.4 mmol of heavy metal ions per gram of composite particles in the case of the composite particles from example 4.

### Example 6

In a similar manner to example 1, composite particles (content of FeOₓ nanoparticles of 15% by weight) having amino functionalization (—NH₂) were prepared. The matrix components are tetraethoxysilane and 3-aminopropyltriethoxysilane in a molar ratio of 1:1. Prehydrolysis of the sol for 24 h and 192 h results in average particle sizes of 231 nm (80% of the composite particles in the 180 nm-335 nm size range) and 1.38 μm (80% of the composite particles in the 1.08 nm-1.74 μm size range) respectively. The isoelectric point is in the pH range from 7.2 to 7.8. The specific magnetization of the superparamagnetic composite particles for a content of FeOₓ single-domain particles of 15% by weight is 11.2 EMU/g (specific magnetization of FeOₓ single-domain particles 70 EMU/g).

### Example 7

In a similar manner to example 6, composite particles were prepared using 21.32 g of a Fe₃O₄ suspension having 6.6% by weight solids content with the addition of 4 g of prehydrolyzed sol (265 h at 60° C.). This results in composite particles having an average particle size of 235 nm (80% of the composite particles in the 185 nm-425 nm size range) and a specific magnetization of 21.4 EMU/g.

### Example 8

In a similar manner to example 6, composite particles were prepared using 21.15 g of a suspension of unmodified Fe₃O₄ having 5.75% by weight solids content with the addition of 7.5 g of prehydrolyzed sol (>3 months at 60° C.). This results in an average size of the composite particles of 1.58 μm (80% of the particles in the 1.33 μm-1.90 μM range). The specific magnetization is found to be 20.2 EMU/g and the isoelectric point of the composite particles is at pH 8.6.

### Example 9

In a similar manner to example 1, composite particles having nitrile functionalization, —C≡N, were prepared. The matrix components are tetraethoxysilane and 2-cyanoethyltrimethoxysilane in a molar ratio of 1:1. The sol was prehydrolyzed at 60° C. for 24 h. The synthesis results in superparamagnetic composite particles having a content of FeOₓ nanoparticles of 15% by weight. The average size of the composite particles is 145 nm (80% of the composite particles in the 115 nm-260 nm size range) and the isoelectric point is at pH 7.9.

### Example 10

10.48 ml of deionized water were added dropwise to 34.52 g of tetraethoxysilane and 37.06 g of N-(2-aminoethyl)-3-aminopropyltrimethoxysilane in 32 ml of ethanol and the sol was treated thermally at 60° C. for 24 h. To the sol were added 140 ml of a suspension of FeOₓ nanoparticles (solids content 4.42% by weight) and the alcohol was subsequently removed by rotary evaporation. 20 g of the sol were stirred into an emulsion having W/O=0.2 and subjected to emulsion evaporation and stirred in the hot solvent at 160° C. for 3 h. This gave composite particles having average sizes of d₅₀=150 nm (d₁₀=110 nm; d₉₀=210 nm). The isoelectric point of the particles is at pH 9.8.

### Example 11

100 mg of amino-functionalized composite particles (average diameter in methanol: 1.2 μm) were suspended in 6 ml of solvent (methanol:H₂O:acetic acid (c=0.2 mol/l)=4:1:1 (v/v/v)) and treated with ultrasound for 3 min. Separately, 89 mg of β-alanine (spacer) were dissolved in a mixture of 1 ml of H₂O and 1 ml of acetic acid (c=2 mol/l) and then 2 ml of methanol were added. This solution was added with stirring to the suspension of the composite particles. Finally, 207 mg of N,N′-dicyclohexylcarbodiimide (DCC) were added and the mixture was stirred at room temperature for 48 h. For processing, the particles were washed repeatedly with methanol, dialyzed against water, isolated by magnetic separation and taken up in 40 ml of methanol.

267 mg of urease were dissolved in 15 ml of solvent (methanol:H₂O:acetic acid (c=2 mol/l)=5:5:1 (v/v/v)) and added to the particle suspension. Separately, 333 mg of N,N′-dicyclohexylcarbodiimide (DCC) were dissolved in 3 ml of methanol and this solution was added dropwise with stirring at room temperature to the particle suspension. After 2 h and after 4 h, in each case 333 mg of DCC in 3 ml of methanol were again added dropwise, and the mixture was stirred at room temperature for a further 5 d. For processing, the composite particles (average diameter in methanol: 3.8 μm) were washed with methanol and dialyzed against water.

The resulting composite particles were used in aqueous suspension at pH 7 to decompose urea. The carbon dioxide thus formed was passed into Ba (OH) 2 solution and detected by precipitation of BaCO₃.

1-10. (canceled)

11. A process for preparing composite particles which comprise superparamagnetic iron oxide particles incorporated in a polysiloxane matrix comprising at least one functional group, the process comprising (a) providing an aqueous-organic emulsion which comprises superparamagnetic iron oxide particles having an average particle diameter of less than 30 nm and a precondensate obtained from one or more hydrolyzable silane compounds, (b) condensing the precondensate to form composite particles which comprise the iron oxide particles incorporated in a polysiloxane matrix, and (c) optionally, reacting the polysiloxane matrix with one or more organic compounds, at least one of (i) said precondensate and (ii) said one or more organic compounds comprising the at least one functional group.

12. The process of claim 11, wherein the polysiloxane matrix comprises a polyorganosiloxane matrix.

13. The process of claim 11, wherein the one or more hydrolyzable silane compounds comprise at least one tetraalkoxysilane.

14. The process of claim 11, wherein at least one of the one or more hydrolyzable silane compounds comprises at least one functional group.

15. The process of claim 14, wherein the at least one functional group is selected from amino, alkyl-substituted amino, carboxyl, carboxylate, epoxy, mercapto, mercaptide, cyano, hydroxyl, and ammonium groups.

16. The process of claim 11, wherein the at least one functional group is selected from amino, alkyl-substituted amino, carboxyl, carboxylate, epoxy, mercapto, mercaptide, cyano, hydroxyl, ammonium groups and combinations thereof.

17. The process of claim 13, wherein the one or more hydrolyzable silane compounds comprise at least one of γ-aminopropyltriethoxysilane, N-(2-aminoethyl)-3-aminopropyltrimethoxysilane, N-(trimethoxysilylpropyl)-ethylenediaminetriacetic acid and a salt thereof, 2-cyanoethyltrimethoxysilane and N-(trimethoxysilyl-propyl)-N,N,N-trimethylammonium chloride.

18. The process of claim 11, wherein the one or more organic compounds comprise at least one hydrolyzable silane compound which comprises at least one functional group selected from amino, alkyl-substituted amino, carboxyl, carboxylate, epoxy, mercapto, mercaptide, cyano, hydroxyl, and ammonium groups.

19. The process of claim 11, wherein the one or more organic compounds comprise at least one of an enzyme, protein, antibody, chemotherapeutic, carbohydrate and organic polymer.

20. The process of claim 11, wherein the superparamagnetic iron oxide particles have an average particle diameter of not more than 20 nm.

21. The process of claim 20, wherein the superparamagnetic iron oxide particles comprise Fe₃O₄.

22. The process of claim 12, wherein the superparamagnetic iron oxide particles comprise at least one of magnetite and maghemite.

23. The process of claim 11, wherein the superparamagnetic iron oxide particles comprise surface-modified iron oxide particles.

24. The process of claim 23, wherein the surface-modified iron oxide particles comprise iron oxide particles modified with at least one silane compound having at least one functional group.

25. The process of claim 24, wherein the at least one functional group comprises an amino group.

26. The process of claim 11, wherein the process further comprises recovering the composite particles obtained after (b) or (c).

27. The process of claim 11, wherein the composite particles have an average particle diameter of not more than 10 μm.

28. The process of claim 11, wherein (a) comprises combining one of (1) an aqueous suspension of the iron oxide particles and (2) an aqueous-organic emulsion comprising the iron oxide particles in an aqueous phase thereof with a sol obtained by hydrolyzing and precondensing one or more hydrolyzable silane compounds in an organic solvent and in the presence of one of an acid and a base, thereby forming a mixture of the precondensate and the iron oxide particles in an aqueous-organic liquid and, if necessary, converting the aqueous-organic liquid to an aqueous-organic emulsion.

29. The process of claim 28, wherein the organic solvent comprises an alcohol.

30. The process of claim 28, wherein hydrolyzing and precondensing is carried out in the presence of an acid.

31. The process of claim 11, wherein the aqueous-organic emulsion comprises a microemulsion.

32. The process of claim 11, wherein (b) comprises an emulsion evaporation.

33. Composite particles obtainable by the process of claim 11.

34. Composite particles which comprise superparamagnetic iron oxide particles having an average particle diameter of less than 30 nm incorporated in a polysiloxane matrix comprising one or more functional groups.

35. The composite particles of claim 34, wherein the superparamagnetic iron oxide particles have an average particle diameter of from 5 nm to 20 nm.

36. The composite particles of claim 34, wherein the composite particles have an average particle diameter of not more than 10 μm.

37. The composite particles of claim 35, wherein the composite particles have an average particle diameter of from 100 nm to 2 μm.

38. The composite particles of claim 36, wherein the composite particles have an average particle diameter of not more than 2 μm.

39. The composite particles of claim 34, wherein the superparamagnetic iron oxide particles comprise at least one of magnetite and maghemite.

40. The composite particles of claim 35, wherein the superparamagnetic iron oxide particles comprise surface-modified iron oxide particles.

41. The composite particles of claim 40, wherein the surface-modified iron oxide particles comprise particles modified with at least one silane compound having at least one functional group.

42. The composite particles of claim 34, wherein the one or more functional groups are independently selected from amino, alkyl-substituted amino, carboxyl, carboxylate, epoxy, mercapto, mercaptide, cyano, hydroxyl, and ammonium groups.

43. The composite particles of claim 34, wherein the composite particles further comprise at least one of an enzyme, protein, antibody, chemotherapeutic, carbohydrate and organic polymer linked thereto.

44. The composite particles of claim 34, wherein the composite particles have an iron oxide content of about 15% by weight.

45. The composite particles of claim 34, wherein the composite particles have a density of 1.7 g/cm³.

46. The composite particles of claim 34, wherein the composite particles have a specific magnetization of from 11.2 to 21.4 EMU/g.

47. Composite particles which comprise superparamagnetic iron oxide particles incorporated in a polyorganosiloxane matrix comprising one or more functional groups, wherein the composite particles have an average particle diameter of from 100 nm to 2 μm, the iron oxide particles comprise at least one of magnetite and maghemite and have an average particle diameter of from 5 nm to 20 run, and the one or more functional groups are independently selected from amino, alkyl-substituted amino, carboxyl, carboxylate, epoxy, mercapto, mercaptide, cyano, hydroxyl, and ammonium groups.

48. The composite particles of claim 47, wherein the superparamagnetic iron oxide particles comprise iron oxide particles which are surface-modified with at least one silane compound having at least one functional group.

49. The composite particles of claim 47, wherein the composite particles have an iron oxide content of about 15% by weight.

50. The composite particles of claim 47, wherein the composite particles have a specific magnetization of from 11.2 to 21.4 EMU/g.

51. Support particles for organic syntheses carried out on solid supports, wherein said support particles comprise the composite particles of claim 34.

52. A magnetic separation process for separating at least one component of a mixture from one or more other components of the mixture, wherein the process comprises adding the composite particles of claim 34 to the mixture and selectively combining said at least one component with the composite particles and separating the composite particles having the at least one component combined therewith by subjecting the mixture to a magnetic field.

53. The process of claim 52, wherein the at least one component comprises a heavy metal ion.

54. A dry powder which comprises the composite particles of claim 34.
